(19) United States
(12) Patent Application Publication
(10) Pub. No.: US 2023/0166041 A1
(43) Pub. Date: Jun. 1, 2023

(54) DELIVERY DEVICE FOR HEMOSTATIC COMPOSITIONS

(71) Applicant: Cresilon, Inc., Brooklyn, NY (US)

(72) Inventors: Omar AHMAD, Brooklyn, NY (US); Jonathan LEE, Brooklyn, NY (US); Matthew SWIFT, Detroit, MI (US); Joseph LANDOLINA, Brooklyn, NY (US)

(21) Appl. No.: 17/881,183

(22) Filed: Aug. 4, 2022

Related U.S. Application Data
(63) Continuation of application No. 15/920,362, filed on Mar. 13, 2018, now abandoned.

(51) Int. Cl. A61M 5/30; A61M 5/315; A61K 9/00; A61K 31/715; A61M 5/19; A61M 5/31

(52) U.S. Cl. CPC A61M 5/30; A61M 5/31513; A61K 9/0019; A61K 31/715; A61M 5/19; A61M 5/3134

(57) ABSTRACT

This disclosure relates to devices and kits for delivery of a fluid. In some embodiments, the fluid is useful in facilitating and maintaining hemostasis.

### CROSS-REFERENCE TO RELATED APPLICATIONS

This application is a continuation of U.S. patent application Ser. No. 15/920,362, filed Mar. 13, 2018. The contents of the aforementioned applications are hereby incorporated by reference.

### BACKGROUND

This disclosure relates to devices and kits for delivery of a fluid, and particularly for delivery of a fluid useful in facilitating and maintaining hemostasis at a wound site.

### BACKGROUND

Hemostasis is a complex, multi-stage mechanism involving an orchestrated effort on the part of many cell types and scaffold formations to begin production of an initial platelet plug at the site of a wound, and then develop a fully mature clot capable of arresting blood flow. Hemostasis is usually divided into three phases: primary hemostasis, the coagulation cascade, and fibrinolysis. Initially, a platelet plug is formed as a response to exposed endothelial cells at a compromised surface, after platelets adhere to collagen fibers surrounding the compromised surface. Exposure to collagen “activates” the platelets, prompting them to release coagulation factors that allow for the coagulation cascade to progress. The process ends in the cleavage of fibrinogen by thrombin to form the foundational material for a clot, known as fibrin.

One challenge in the treatment of bleeding surfaces at a wound site is the rapid and uniform delivery of hemostatic compositions directly to the bleeding surfaces. Conventional delivery devices, such as syringes, typically comprise Luer-lock style needle adapters to which a needle or needle-like applicator is coupled. The small bore sizes often associated with such needles or needle-like applicators may prevent rapid deployment of the fluid disposed within the syringe. Moreover, the threaded fittings used to couple such needles or needle-like applicators to the syringe are typically disposed within the interior of the Luer-lock style needle adapters. This can result in excess shearing of the fluid as it is being dispensed/deposited.

### SUMMARY

Provided herein, in one embodiment, is a delivery device comprising a barrel comprising opposing first and second ends, and a first interior chamber positioned therebetween, wherein the first interior chamber is configure to receive a fluid. The delivery device additionally comprises a plunger comprising a stopper, an end portion, and a shaft extending therebetween, wherein the stopper and one or more portions of the shaft are configured to be disposed within the first interior chamber of the barrel, and wherein the plunger is configured to move along the longitudinal axis of the barrel to displace fluid disposed therein. The delivery device further comprises an applicator region proximal the first end of the barrel, the applicator region comprising a second interior chamber in fluid communication with the first internal channel of the barrel, and a connector on an exterior surface of the applicator region.

In some embodiments of the delivery device, a diameter of the second interior chamber of the applicator region is at least about 50% of a diameter of the first interior chamber of the barrel. In some embodiments, the diameter of the second interior chamber of the applicator region is from about 50% to about 90% of the diameter of the first interior chamber of the barrel.

In some embodiments of the delivery device, a diameter of the second interior chamber of the applicator region is at least about 5 mm. In some embodiments, the diameter of the second interior chamber of the applicator region is at least about 6.5 mm. In some embodiments, the diameter of the second interior chamber of the applicator region is from about 6 mm to about 10 mm.

In some embodiment of the delivery device s, the connector on the exterior surface of the applicator region comprises a plurality of threads.

In some embodiments of the delivery device, the applicator region is configured for needle-less application of the fluid.

In some embodiments of the delivery device, the delivery device further comprising a cap configured to surround one or more portions of the applicator region, the cap comprising a second connector complementary to the connector of the applicator region. In some embodiments, the connector of the applicator region comprises a plurality of male threads, and the second connector of the cap comprises a plurality of female threads. In some embodiments of the delivery device, a cross-sectional area of the end portion of the plunger is larger than a cross-sectional area of the first interior chamber of the barrel. In some embodiments, the stopper of the plunger comprises a substantially flat surface configured to contact the first end of the barrel. In some embodiments, the stopper of the plunger is deformable such that the stopper of the plunger is moveable within the first interior chamber. In some embodiments, the stopper further comprises an engagement member adapted to contact a retaining member at or near the second end of the first interior chamber preventing separation of the plunger from the first interior chamber.

In some embodiments, the barrel further comprises a grip region proximal the second end thereof, and which extends substantially perpendicular to the barrel.

Also provided herein, in one embodiment, is a kit comprising a delivery device as described herein, and a fluid to be dispensed from the delivery device.

In some embodiments of the kit, the aforementioned fluid is a hemostatic composition. In some embodiments, the fluid is VETIGEL™. In some embodiments, the fluid is disposed within the first interior chamber of the delivery device.

In some embodiments of the kit, the kit further comprises a sealed package in which the delivery device and the fluid is enclosed.

Also provided herein, in one embodiment, is a needleless delivery device, comprising: a barrel comprising opposing first and second ends, and a first interior chamber positioned therebetween, and a fluid disposed within the first interior chamber. The needless delivery device also comprises a plunger comprising a stopper, an end portion, and a shaft extending therebetween, wherein the stopper and one or more portions of the shaft are configured to be disposed within the first interior chamber of the barrel, and wherein the plunger is configured to move along the longitudinal axis of the barrel to displace the fluid disposed therein. The needleless delivery device further comprises an applicator region proximal the first end of the barrel, where the applicator region comprises a second interior chamber in fluid communication with the first internal channel of the barrel, and a connector solely on an exterior surface of the applicator region.

In some embodiments of the needleless delivery device, the diameter of the second interior chamber of the applicator region is from about 50% to about 90% of the diameter of the first interior chamber of the barrel. In some embodiments, the diameter of the second interior chamber of the applicator region is from about 6 mm to about 10 mm.

In some embodiments of the needleless delivery device, the connector comprises a plurality of threads.

In some embodiments, the needleless delivery device further comprise a cap configured to surround one or more portions of the applicator region, wherein the cap comprises a second connector complementary to the connector of the applicator region.

In some embodiments of the needleless delivery device, the fluid disposed within the first interior chamber is a hemostatic composition. In some embodiments, the fluid is VETIGEL™.

In some embodiments of the needleless delivery device, the plunger further comprises one or more engagement members positioned at or near the stopper and adapted to contact one or more retaining members at or near the second end of the first interior chamber preventing separation of the plunger from the first interior chamber.

### BRIEF DESCRIPTION OF THE DRAWINGS

Exemplary and non-limiting embodiments of the inventions may be more readily understood by referring to the accompanying drawings, in which:

FIG. 1 shows a delivery device comprising at least a barrel, a plunger, and an applicator region, according to one embodiment.

FIG. 2 is a cross-sectional, top down view of a grip region located on the barrel of the delivery device of FIG. 1.

FIG. 3 is a side view of the plunger of the delivery device of FIG. 1.

FIG. 4 is a top down view of the end portion of the plunger of the delivery device of FIG. 1.

FIGS. 5A-5B show a side view and a cross-sectional, side view, respectively, of the barrel of the delivery device of FIG. 1.

### DETAILED DESCRIPTION

Described herein are devices and kits for delivery of a fluid, and particularly for delivery of a fluid useful in facilitating and maintaining hemostasis at a wound site. In some embodiments, the delivery devices and kits thereof comprise a large bore applicator region, which not only allows for more rapid delivery of the fluid, but also for a more uniform, flowable dose of the fluid to a targeted area. In some embodiments, the large bore applicator region need not rely on interior threaded fittings, thereby preventing excess shearing of the fluid as it is being dispensed/delivered.

### 1. Delivery Devices

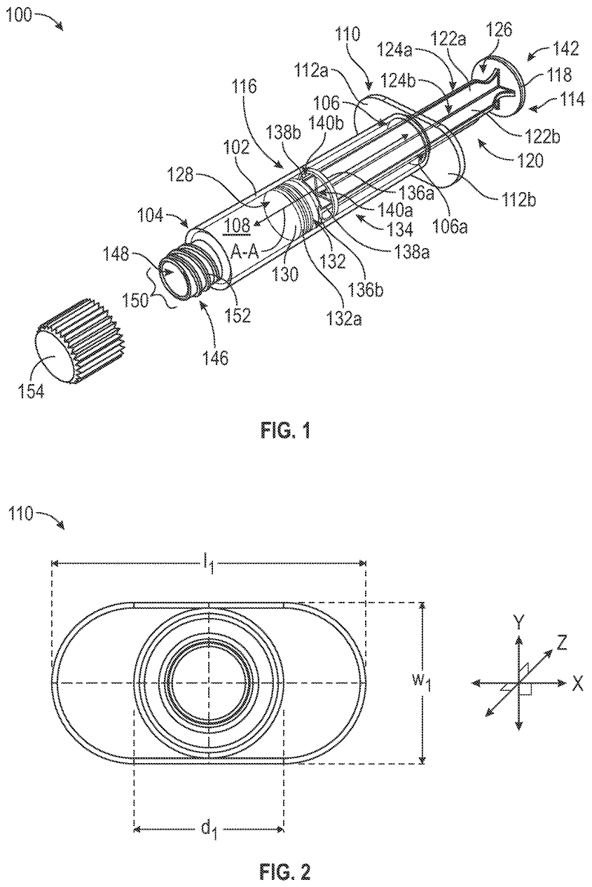

Referring now to FIG. 1, an isometric view of a delivery device (e.g., syringe) 100 is shown in accordance with one embodiment. The delivery device 100 or components/features thereof may be implemented in combination with, or as an alternative to, other devices/features/components described herein, such as those described with reference to other embodiments and figures. The delivery device 100 may additionally be utilized in any of the methods for making and/or using such devices/components/features described herein. The delivery device 100 may also be used in various applications and/or permutations, which may or may not be noted in the illustrative embodiments described herein. For instance, delivery device 100 may include more or less features/components than those shown in FIG. 1, in some embodiments. Moreover, the delivery device 100 is not limited to the size, shape, number of components, etc. specifically shown in FIG. 1.

As shown in FIG. 1, the delivery device 100 comprises a barrel 102 that has opposing first and second ends 104, 106, and a first interior chamber 108 positioned therebetween. The barrel 102 may comprise a generally cylindrical shape, with a longitudinal axis extending along the line denoted A-A in FIG. 1.

The first interior chamber 108 is a hollow cavity configured to receive a fluid. As used herein in some embodiments, the term “fluid” may refer to a flowable substance capable of being passed through and dispensed from the delivery device 100 in a controlled manner, such as a liquid, gel, solution, suspension, etc. As discussed in greater detail infra, the fluid may comprise a biocompatible polymeric composition in some embodiments. In some embodiments, the biocompatible polymeric composition is in the form of a gel and configured to facilitate and maintain hemostasis. In some embodiments, the fluid is VETIGEL™.

In some embodiments, the volumetric capacity of the first interior chamber 108 may be in a range from about 3 mL to about 25 mL, or from about 5 mL to about 10 mL. In some embodiments, the volumetric capacity of the first interior chamber 108 may be in a range between and including any two of the following: about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, and about 25 mL. In some embodiments, the volumetric capacity of the first interior chamber 108 may be about 5 mL.

In some embodiments, the outer/exterior surface of the barrel 102 may comprise one or more markings. For instance, in some embodiments, the outer surface of the barrel 102 may comprise markings indicating the volumetric capacity of the first interior chamber 108. In some embodiments, the outer surface of the barrel 102 may comprise an identification (e.g., a chemical, or known brand name) of the fluid dispensed therein. In some embodiments, the outer surface of the barrel 102 may comprise any or all of the foregoing markings, or other relevant markings as would become apparent to one skilled in the art upon reading the present application.

In some embodiments, the barrel 102 may comprise a grip region (or flange) 110 proximal to the second end 106 thereof and extending radially from the outer surface of the barrel 102. In some embodiments, this grip region 110 may be oriented substantially perpendicular to the longitudinal axis of the barrel 102. In some embodiments, the grip region 110 may have an ergonomic shape so as to allow a human to comfortably grip the barrel 102 of the delivery device 100. For instance, as shown in the embodiment of FIG. 1, the grip region 110 may have at least two protruding regions 112a, 112b radially extending some distance from the outer surface of the barrel 102. In some embodiments, each of the protruding regions 112a, 112b may independently extend a distance sufficient to allow at least one finger of a human hand to curl about or otherwise grip at least one of the protruding regions 112a, 112b.

In some embodiments, the second end 106 may further comprise a retaining member 106a, e.g., a retaining lip or other formation protruding into the barrel 102 at or near the second end 106. FIG. 1 illustrates one embodiment, in which retaining member 106a substantially encircles the interior-facing area (of barrel 102) at the second end 106 thereof. However, in some embodiments, retaining member 106a may comprise one or more inward protruding formations or multiple retaining members may be implemented, e.g., a single tab or protrusion, two opposing protrusions, a series of protrusions, or other configuration that effectively prevents plunger 114 (described in greater detail below) from completely exiting the barrel 102 at the second end 106 thereof. That is, retaining member 106a may engage an opposing member or area of plunger 114 to prevent or provide resistance that takes a relatively significant amount of pulling force (on plunger 114) to overcome. In some embodiments, this amount of pulling force is greater than the amount of force a user would apply to move plunger 114 through barrel 102 to dispense a material, e.g., fluid, therein. It should be understood that retaining member 106a may be formed as part of the second end 106 of barrel 102, or may be formed separately, using the same or different material(s) as that used to form barrel 102.

FIG. 2 provides a cross-sectional, top down view of the grip region 110 for reference. In some embodiments, a longitudinal length, lᵢ, of the grip region 110, as measured along the x-axis of FIG. 2, may be in a range from about 10 mm to about 50 mm. In some embodiments, the longitudinal length, lᵢ, of the grip region 110 may be in range between and including any two of the following: about 10 mm, about 13 mm, about 15 mm, about 20 mm, about 22 mm, about 24 mm, about 26 mm, about 28 mm, about 30 mm, about 32 mm, about 34 mm, about 36 mm, about 38 mm, about 40 mm, about 42 mm, about 44 mm, about 46 mm, about 48 mm, and about 50 mm. In some embodiments, the longitudinal length, lᵢ, of the grip region 110 may be about 32.6 mm.

In some embodiments, the width, w₁, of the grip region 110, as measured along the y-axis of FIG. 2, may be in a range from about 10 mm to about 50 mm. In some embodiments, the width, w₁, of the grip region 110 may be in range between and including any two of the following: about 10 mm, about 13 mm, about 15 mm, about 20 mm, about 22 mm, about 24 mm, about 26 mm, about 28 mm, about 30 mm, about 32 mm, about 34 mm, about 36 mm, about 38 mm, about 40 mm, about 42 mm, about 44 mm, about 46 mm, about 48 mm, and about 50 mm. In some embodiments, the width, w₁, of the grip region 110 may be about 16.6 mm.

In some embodiments, the width, w₁, of the grip region 110 may be about equal to or less than the longitudinal length, l₁, of grip region 110. In some embodiments, the width, w₁, of the grip region 110 may be less than the longitudinal length, l₁, of grip region 110. In some embodiments, the width, w₁, of the grip region 110 may about equal to the longitudinal length, l₁, of grip region 110.

In some embodiments, the width of the grip region 110 may be about equal to the outer diameter, d₁, of the barrel 102. In some embodiments, the width of the grip region 110 may be greater than the outer diameter, d₁₀, of the barrel 102, but less than the longitudinal length, l₁, of the grip region 110.

In some embodiments, the outer diameter, d₁, of the barrel 102 may be in a range from about 10 mm to about 50 mm. In some embodiments, the outer diameter, d₁, of the barrel 102 may be in range between and including any two of the following: about 10 mm, about 13 mm, about 15 mm, about 20 mm, about 22 mm, about 24 mm, about 26 mm, about 28 mm, about 30 mm, about 32 mm, about 34 mm, about 36 mm, about 38 mm, about 40 mm, about 42 mm, about 44 mm, about 46 mm, about 48 mm, and about 50 mm. In some embodiments, the outer diameter, d₁, of the barrel 102 may be about 14.6 mm.

As shown in the embodiments of FIGS. 1-2, the grip region 110 may have a substantially stadium shape, where the at least two protruding regions 112a, 112b have a substantially semicircular shape. The grip region 110, however, is not limited to the configuration shown in FIGS. 1-2, and may comprise an overall circular shape, oval shape, elliptical shape, square shape, rectangular shape, irregular or asymmetrical shape, or any suitable shape as would be apparent to one skilled in the art upon reading the present disclosure.

In some embodiments, the grip region 110 of FIGS. 1-2 may be a monolithic structure (e.g., prepared from a single piece of material). In some embodiments, however, the at least two protruding 112a, 112b may be prepared as separate components, each independently connected/coupled to the outer surface of the barrel 102.

With continued reference to FIG. 1, the delivery device 100 comprises a plunger 114. The plunger 114 comprises a stopper 116, an end portion 118, and a shaft 120 extending therebetween. The stopper 116 and one or more portions of the shaft 120 are configured, e.g., are of suitable dimensions, to be disposed within the first interior chamber 108 of the barrel 102. Such a configuration allows one or more portions of the plunger 114 to move along the longitudinal axis of the barrel 102 toward the first end 104 thereof, thereby displacing a fluid disposed therein. Such a configuration also allows the plunger 114 to move along the longitudinal axis of the barrel 102 toward the second end 106 thereof, thereby withdrawing a fluid into the first interior chamber 108 of the barrel 102.

In some embodiments, the shaft 120 may comprise one or more struts 122 (beams) extending between the stopper 116 and the end portion 118 of plunger 114. In the embodiment of FIG. 1, the shaft 120 may comprise four such struts (beams) 122a, 122b, 122c, 122d, each comprise an outer edge 124a and an inner edge 124b. The struts 122a, 122b, 122c, 122d may be coupled to one another substantially along their respective inner edges 124b. Further, in some embodiments, the struts 122a, 122b, 122c, 122d may be oriented substantially perpendicular to one another. Further still, In some embodiments, the outer edge 124a of each of the struts 122a, 122b, 122c, 122d may comprise a generally concave region 126 near the end portion 118 of the plunger 114. It should be noted that strut 122d is not shown due to perspective view limitations of the figures included herein. However, one skilled in the art would understand that strut 122d may be oriented perpendicular to, e.g., struts 122a and 122c, and in the same plane as strut 122b.

In some embodiments, the shaft 120 comprising struts 122a, 122b, 122c, 122d may be a monolithic structure (e.g., prepared from a single piece of material). In some embodiments, however, each of the struts 122a, 122b, 122c, 122d may be prepared as separate components, which are connected/coupled together to form the shaft 120.

In some embodiments, the plunger 114 may be dimensioned such that substantially an entire longitudinal length of the shaft 120 may extend within the first interior chamber 108 of the barrel 102. In some embodiments, shaft 120 may be inserted within the first interior chamber 108 of the barrel 102 up to about the generally concave regions 126 of the struts 122.

It is of note that the shaft 120 is not limited to the configuration and/or number of components (e.g., struts) shown in FIG. 1. For instance, in some embodiments, the shaft 120 may comprise more or less struts than shown in FIG. 1. In some embodiments, the shaft 120 may have the form of a solid cylinder or tube, or hollow cylinder or tube, extending between the stopper 116 and the end portion 118 of plunger 114. In some embodiments, the shaft 120 may have any suitable shape complementary to the barrel 102 such that one or more portions of the shaft may be inserted and/or moved within the first interior chamber 108 of the barrel 102.

In some embodiments, the stopper 116 of the plunger 114 may comprise a surface 128 configured to contact the first end 104 of the barrel 102. In some embodiments, the surface 128 may be substantially flat and oriented substantially perpendicular to the longitudinal axis of the barrel 102.

As shown in FIG. 1, the stopper 116 may comprise a seal portion 130 that terminates at the surface 128. One or more portions of the side 132 of the seal portion 130 may be in contact with an inner surface of the barrel 102. The seal portion 130 may form a fluid and/or air tight seal between the stopper 116 and the inner surface of the barrel 102. The seal portion 130 may additionally be configured to slide along the inner surface of the barrel 102 without compromising the integrity of the fluid and/or air tight seal.

In some embodiments, the side 132 of the seal portion 130 may comprise one or more ridges (or ribs) (e.g., between each of which is a recessed groove), which are configured to form a tight, slideable engagement with the inner surface of the barrel 102.

As noted above, the second end 106 of barrel 102 may comprise a retaining member 106a, e.g., a retaining lip or other formation protruding into the barrel 102 at or near the second end 106. As also discussed above, retaining member 106a may engage some opposing member or area of plunger 114. In some embodiments, the opposing member or area of plunger 114 may be implemented as part of side 132 of the seal portion 130. That is, side 132 of the seal portion 130 may further comprise one or more engaging members 132a for resistively contacting or engaging retaining member 106a. In some embodiments, engaging member 132a may be a circumferential protrusion adapted to abut against a side of retaining member 106 facing the interior of barrel 102. In some embodiments, engaging member 132a need not comprise a completely circumferential protrusion, but may comprise one or more protrusion, tabs, or other formations or regions that engage retaining member 106a. For example, a plurality of engaging members 132a may each comprise protruding regions located periodically or aperiodically about the circumference of side 132. In some embodiments, engaging member 132a may be formed from/out of the same material(s) as that used to form seal portion 130. In some embodiments, engaging member 132a may be formed from a different material(s) and securedly attached to seal portion 130 at side 132.

In operation, a user may impart a pulling force to plunger 114, the pulling force being in the direction of the longitudinal axis of barrel 102 along line A-A. Upon engaging member 132a engaging or otherwise coming into contact with retaining member 106a, the user would have to impart significant additional pulling force to plunger 114 to separate plunger 114 from barrel 102. The amount of additional pulling force that would be needed to separate plunger 114 from barrel 102 can vary. One of ordinary skill in the art would understand that varying the size, thickness, and/or other relevant dimension(s) of either or both of retaining member 106a and engaging member 132a can be adapted to provide a desired amount of resistance opposing the pulling force applied to plunger 114 in order to effectively prevent or make it difficult to separate plunger separate plunger 114 from barrel 102. The strength, deformation characteristics, or other relevant aspect(s) of the material(s) used to form retaining member 106a and/or engaging member 132a may also be varied to achieve the desired amount of resistance.

That is, retaining member 106a may engage an opposing member or area of plunger 114 to prevent or provide resistance that takes a relatively significant amount of pulling force (on plunger 114) to overcome. In some embodiments, this amount of pulling force is greater than the amount of force a user would apply to move plunger 114 through barrel 102 to dispense a material, e.g., fluid, therein.

In some embodiments, the seal portion 130 may comprise an elastomer material. In some embodiments, the seal portion 130 may comprise a thermoplastic elastomer material. Example materials for the seal portion 130 may include, but are not limited to, one or more of natural rubber. acrylate-butadiene rubber, chlorobutyl rubber. cis-polybutadiene, chlorinated polyethylene elastomers, fluorosilicone rubbers, butyl rubbers, silicone rubbers, styrene-butadiene rubbers, tetrafluoroethylene propylene copolymer, polyisobutene, thermoplastic copolyesters, and the like.

As shown in the embodiment of FIG. 1, the stopper 116 may optionally comprise an intermediate structure 134 between the seal portion 130 and the shaft 120. In some embodiments, the intermediate structure 134 may comprise opposing first and second surfaces 136a, 136b, and one or more struts 138 extending therebetween. The first and second surfaces 136a, 136b may be oriented substantially perpendicular to the longitudinal axis of the barrel 102.

In some embodiments, the intermediate structure 134 may comprise four struts 138a, 138b, 138c, 138d, each strut comprising an outer edge 140a and an inner edge 140b. It should be noted that struts 138c and 138d are not illustrated due to perspective view limitations of the figures included herein. The struts 138a, 138b, 138c, 138d may be coupled to one another substantially along their respective inner edges 140b. Moreover, in some embodiments, the struts 138a, 138b, 138c, 138d may be oriented substantially perpendicular to one another. In some embodiments, the struts 138a, 138b, 138c, 138d of the intermediate structure 134 may coincide, e.g., be aligned with, with the struts 122a, 122b, 122c, 122d of the shaft 120.

In some embodiments, the intermediate structure 134 comprising first and second opposing surfaces 136a, 136b and the one or more struts 138 (e.g., struts 138a, 138b, 138c, 138d) may be a monolithic structure (e.g., prepared from a single piece of material). In some embodiments, however, each of the struts 138 may be prepared as separate components, which are connected/coupled.

In some embodiments, the plunger 114 may not include the intermediate structure 134, such that the seal portion 130 may be proximal to, and in contact with, the shaft 120.

In some embodiments, the seal portion 130 and/or the intermediate structure 134 may have a generally circular cross-sectional shape, where the cross-section is taken perpendicular to the longitudinal axis of the barrel 102. In some embodiments, the seal portion 130 and/or the intermediate structure 134 may have a cross-sectional shape that is substantially the same as the cross-sectional shape of the barrel 102.

In some embodiments, the diameter of the seal portion 130 may be about equal to the diameter of the first and second surfaces 136a, 136b of the intermediate structure 134. In some embodiments, the diameter of the seal portion 130 may be greater than the diameter of the first and second surfaces 136a, 136b of the intermediate structure 134. In some embodiments, the diameter of the seal portion 130 may be less than the diameter of the first and second surfaces 136a, 136b of intermediate structure 134.

In some embodiments, the diameter of the seal portion 130 and/or the first and second surfaces 136a, 136b of the intermediate structure 134 may be in a range from about 8 mm to about 20 mm, or from about 10 mm to about 15 mm. In some embodiments, the diameter of the seal portion 130 and/or the first and second surfaces 136a, 136b of the intermediate structure 134 may be in a range between and including any two of the following: about 8 mm, about 9 mm, about 10 mm, about 11 mm, about 12 mm, about 13 mm, about 14 mm, about 15 mm, about 16 mm, about 17 mm, about 18 mm, about 19 mm, and about 20 mm. In some embodiments, the diameter of the seal portion 130 and/or the intermediate structure 134 may be about 12 mm.

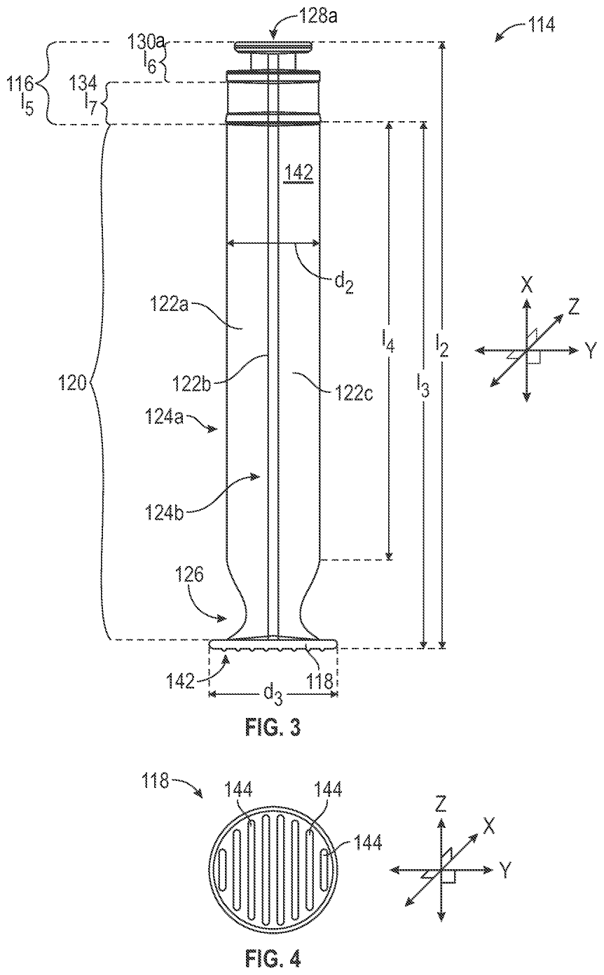

FIG. 3 provides a side view of the plunger 114. In some embodiments, a longitudinal length, l₂, as measured along the x-axis of FIG. 3, of the plunger 114 from the surface 128 of the stopper 116 to the end portion 118 may be in a range from about 50 mm to about 100 mm, or from about 65 mm to about 80 mm. In some embodiments the longitudinal length, l₂, of the plunger 114 from approximately the surface 128 of the stopper 116 to the end portion 118 may be in a range between and including any two of the following: about 50 mm, about 52 mm, about 54 mm, about 56 mm, about 58 mm, about 60 mm, about 62 mm, about 64 mm, about 66 mm, about 68 mm, about 70 mm, about 72 mm, about 74 mm, about 76 mm, about 78 mm, about 80 mm, about 82 mm, about 84 mm, about 86 mm, about 88 mm, about 90 mm, about 92 mm, about 94 mm, about 96 mm, about 98 mm, and about 100 mm. In some embodiments the longitudinal length, l₂, of the plunger 114 from the surface 128 of the stopper 116 to the end portion 118 may be about 74 mm.

It should be understood that plunger 114 as illustrated in FIG. 3 is shown without seal portion 130. That is, FIG. 3 illustrates a seal mounting area 130a, onto which seal portion 130 may be attached (see FIG. 1). Seal mounting area 130a may include a mounting structure 128a that can comprise a mounting button or stud atop a second intermediate structure that may comprise struts (similar to intermediate structure 134). An annular or semi-annular region within an interior area of seal portion 130 may create one or more surfaces configured to contact and/or form around mounting structure 128a, such that seal portion 130 remains affixed to plunger 114 during use.

In some embodiments, a longitudinal length, l₃, of the shaft 120 may be in a range from about 40 mm to about 80 mm. In some embodiments, a longitudinal length, l₃, of the shaft 120 may be in a range between and including any two of the following: about 40 mm, about 42 mm, about 44 mm, about 46 mm, about 48 mm, about 50 mm, about 52 mm, about 54 mm, about 56 mm, about 58 mm, about 60 mm, about 62 mm, about 64 mm, about 66 mm, about 68 mm, about 70 mm, about 72 mm, about 74 mm, about 76 mm, about 78 mm, and about 80 mm. In some embodiments, a longitudinal length, l₃, of the shaft 120 may be about 65 mm.

In some embodiments, a longitudinal length, l₄, of the shaft 120 excluding the generally concave regions 126 of the struts 122 may be in a range from about 40 mm to about 80 mm. In some embodiments, a longitudinal length, l₄, of the shaft 120 excluding the generally concave regions 126 of the struts 122 may be in a range between and including any two of the following: about 40 mm, about 42 mm, about 44 mm, about 46 mm, about 48 mm, about 50 mm, about 52 mm, about 54 mm, about 56 mm, about 58 mm, about 60 mm, about 62 mm, about 64 mm, about 66 mm, about 68 mm, about 70 mm, about 72 mm, about 74 mm, about 76 mm, about 78 mm, and about 80 mm. In some embodiments, a longitudinal length, l₄, of the shaft 120 excluding the generally concave regions 126 of the struts 122 may be about 65 mm.

In some embodiments, a longitudinal length, l₅, of the stopper 116 may be in a range from about 3 mm to about 15 mm. In some embodiments, a longitudinal length, l₅, of the stopper 116 may be in a range between and including any two of the following: about 3 mm, about 4 mm, about 5 mm, about 6 mm, about 7 mm, about 8 mm, about 9 mm, about 10 mm, about 11 mm, about 12 mm, about 13 mm, about 14 mm, and about 15 mm. In some embodiments, a longitudinal length, l₅, of the stopper 116 may be about 9 mm.

In some embodiments, a longitudinal length, l₆, of the seal portion 130 of the stopper 116 may be in a range from about 2 mm to about 5 mm. In some embodiments, a longitudinal length, l₆, of the seal portion 130 may be in a range between and including any two of the following: about 2 mm, 3 mm, about 4 mm, and about 5 mm. In some embodiments, a longitudinal length, l₆, of the seal portion 130 may be about 3.7 mm.

In some embodiments, a longitudinal length, l₇, of the intermediate structure 134 of the stopper 116 may be in a range from about 3 mm to about 10 mm. In some embodiments, a longitudinal length, l₇, of the intermediate structure 134 may be in a range between and including any two of the following: about 3 mm, 4 mm, about 5 mm, about 6 mm, about 7 mm, about 8 mm, about 9 mm, and about 10 mm. In some embodiments, a longitudinal length, l₇, of the intermediate structure 134 may be about 5.2 mm.

In some embodiments, a diameter, d₂, of the shaft 120 (and/or the diameter of the seal portion 130 and/or the intermediate structure 134) may be in a range from about 7 mm to about 20 mm. In some embodiments, a diameter, d₂, of the shaft 120 (and/or the diameter of the seal portion 130 and/or the intermediate structure 134) may be in a range between and including any two of the following: about 7 mm, about 8 mm, 9 mm, about 10 mm, about 11 mm, about 12 mm, about 13 mm, about 14 mm, about 15 mm, about 16 mm, about 17 mm, about 18 mm, about 19 mm, and about 20 mm. In some embodiments, a diameter, d₂, of the shaft 120 (and/or the diameter of the seal portion 130 and/or the intermediate structure 134) may be about 12 mm.

As further shown in FIGS. 1 and 3, the plunger 114 may comprise an end portion 118 proximal to, and in contact with, the shaft 120, and opposite to the seal portion 130.

In some embodiments, the end portion 118 may have a cross-sectional area, or diameter, d₃, larger than the diameter of the first interior chamber 108 of the barrel 102. In some embodiments, the diameter, d₃, of the end portion 118 may be in a range from about 10 mm to about 25 mm, provided that said diameter, d₃, is larger than the diameter of the first interior chamber 108. In some embodiments, the diameter, d₃, of the end portion 118 may be in a range between and including any two of the following: about 10 mm, about 11 mm, about 12 mm, about 13 mm, about 14 mm, about 15 mm, about 16 mm, about 17 mm, about 18 mm, about 19 mm, and about 20 mm, provided that said diameter, d₃, is larger than the diameter of the first interior chamber 108.

In some embodiments, the diameter, d₃, of the end portion 118 may be dimensioned at least commensurate with the average width of a finger or thumb or a human hand. For instance, in some embodiments, the end portion 118 may be dimensioned so as to allow for the thumb of a human hand to comfortably contact and press said end portion 118.

In some embodiments, the upper surface 142 of the end portion 118 of the plunger 114 may have one or more ridges 144, as shown, e.g., in the top down view of the end portion 118 of FIG. 4. Such ridges 142 may prevent a human finger or thumb from slipping off the end portion 118. Other similar features, e.g. raised “islands” or dots, may also be present on the upper surface 142 of the end portion 118 in combination with, or as alternative to, the ridges 144.

With continued reference to FIG. 1, the delivery device 100 comprises an applicator region 146 proximal to the first end 104 of the barrel 102, where the applicator region 144 comprises a second interior chamber 148. Similar to the first interior chamber 108, the second interior chamber 148 is hollow and configured to receive a fluid. The applicator region 146 additionally comprises an output region 150 from which the fluid may be dispensed.

The second interior chamber 148 is in fluid communication with the first interior chamber 108, and may therefor receive the fluid originally present therein. For instance, when a fluid is initially dispensed in the first interior chamber 108 and the plunger 114 is depressed/moved toward the first end 104 of the barrel 102, the fluid may travel to the applicator region 146 and ultimately be dispensed from the output region 150 thereof. In some embodiments, the fluid may be originally present in both the first and second interior chambers 108, 148.

In some embodiments, the second interior chamber 148 of the applicator region 146 may have a diameter (bore size) larger than conventional delivery devices (syringes). The large bore size of the applicator region 146 allows for a more uniform, flowable dose of the fluid to be dispensed at a faster rate as compared to conventional delivery devices (syringes) comprising Luer-lock-style tips.

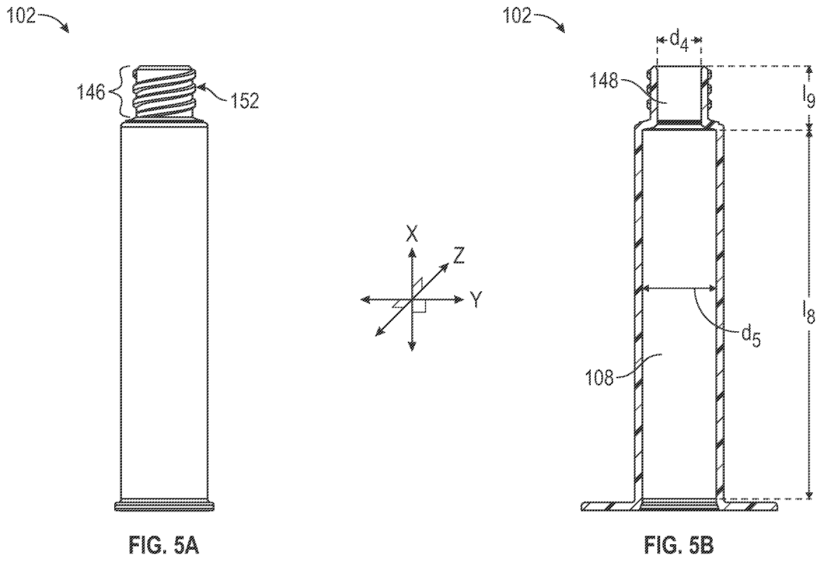

FIGS. 5A-5B respectively show a side view and a cross-sectional, side view of the barrel 102 for reference. In some embodiments, the diameter, d₄, of second interior chamber 148 of the applicator region 146 is at least about 50% of the diameter, d₅, of the first interior chamber 108 of the barrel 102. In some embodiments, the diameter, d₄, of the second interior chamber 148 is from about 50% to about 90% of the diameter, d₅, of the first interior chamber 108.

In some embodiments, the diameter, d₄, of the second interior chamber 148 of the applicator region 146 is at least about 5 mm. In some embodiments, the diameter, d₄, of the second interior chamber 148 is at least about 6.5 mm. In some embodiments, the diameter, d₄, of the second interior chamber 148 is in a range from about 6 mm to about 10 mm. In some embodiments, the diameter, d₄, of the second interior chamber 148 is in a range between and including any two of the following: about 6 mm, about 7 mm, about 8 mm, about 9 mm, and about 10 mm. In some embodiments, the diameter, d₄, of the second interior chamber 148 is about 6 mm or 7 mm. It some embodiments, the material(s) to be dispensed through delivery device 100 is of a constitution such that bore size of the second interior chamber 148 impacts delivery and efficacy of the material(s) in facilitating and maintaining hemostasis at a wound site. For example, in some embodiments, the second interior chamber 148 being in a range from about 6 mm to about 10 mm allows the material(s) dispensed therefrom to re-associate with itself to maintain strength at the wound site.

In some embodiments, the diameter, d₅, of the first interior chamber 108 of the applicator region 146 is from about 8 mm to about 25 mm. In some embodiments, the diameter, d₅, of the first interior chamber 108 may be in a range between and including any two of the following: about 8 mm, 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, and about 25 mm. In some embodiments, the diameter, d₅, of the first interior chamber 108 may be about 12 mm.

In some embodiments, the longitudinal length, l₈, of the first interior chamber 108 of the barrel 102 may be in a range from about 50 mm to about 80 mm, or from about 55 mm to about 70 mm. In some embodiments, the longitudinal length, l₈, of the first interior chamber 108 may be in a range between and including any two of the following: about 50 mm, about 52 mm, about 54 mm, about 56 mm, about 58 mm, about 60 mm, about 62 mm, about 64 mm, about 66 mm, about 68 mm, about 70 mm, about 72 mm, about 74 mm, about 76 mm, about 78 mm, and about 80 mm. In some embodiments, the longitudinal length, l₈, of the first interior chamber 108 may be about 64 mm.

In some embodiments, the longitudinal length, l₉, of the second interior chamber 108 of the barrel 102 may be in a range from about 5 mm to about 20 mm. In some embodiments, he longitudinal length, l₉, of the second interior chamber 108 may be in a range between and including any two of the following: about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, and about 20 mm. In some embodiments, the longitudinal length, l₉, of the second interior chamber 108 may be about 10 mm.

As further shown in FIG. 1 and FIGS. 4A-4B, the applicator region 146 comprises connector 152 on an exterior surface of the applicator region 146. In some embodiments, the connector 152 may comprise a plurality of threads. In some embodiments, the connector 152 may comprise a male or female national pipe thread (NPT) type fitting. In some embodiments, the connector 152 may comprise a Luer taper type fitting. In some embodiments, the connector 152 may comprise a female or male Luer taper type fitting. It should be understood that the connecter 152 may comprise other types of fittings adapted to connect to other types of syringe tips including, but not limited to, a curved dental tip, a fenestrated tip, a laparscopic tip, and a Luer adapter tip.

The presence of connector 152 on the exterior surface of the applicator region 146, rather than within the second interior chamber 148 thereof, prevents excess shearing of material (e.g., the aforementioned fluid) as the material is deposited at a given location (e.g., at the site of a wound on a mammalian subject).

In some embodiments, the delivery device 100 may be a needle-less or needle-free device. For instance, in some embodiments, the applicator region 146 of the delivery device 100 may not be configured for connection with a needle or needle-like applicator.

Again with reference to FIG. 1, the delivery device may further comprise a cap 154 configured to surround one or more portions of the applicator region 146. The cap 154 may comprise a second connector (not shown) complementary to the connector of the applicator region 146. For instance, in some embodiments, the connection means of the applicator region 146 and the connection means of the cap 154 may comprise a plurality of complementary male and female threads, respectively (or vice versa). In some embodiments, the cap 154, when secured to the applicator region 146, may provide a tight seal so as to prevent the leakage of fluid present within the delivery device 100.

In some embodiments, the delivery device 100 may be pre-filled and packaged with the fluid. In such instance, the various components of the delivery device 100 may each independently be comprised of a material(s) that is/are chemically and biochemically compatible (or inert), preferably over extended periods of time, with the fluid dispensed therein. Moreover, in some embodiments, the delivery device 100 may need to be sterilized prior to receiving the fluid. According, the various components of the delivery device 100 may each independently be comprised of a material(s) that exhibits physical characteristics suitable to withstand the pressures, temperatures and other forces experienced during sterilization, such as autoclave sterilization. Example materials for such components may include, but are not limited to, plastic materials, glass, combinations thereof, etc.

in sonic embodiments, at. least the barrel 102 and the applicator region 146 may each be independently comprised of a clear transparent material so as to enable the fluid within to be visible. In some embodiments, substantially all the components of the delivery device 100, except the seal portion 130 and/or a fluid dispensed within the device, may be comprised of a clear, transparent material.

### 2. Fluids

The delivery devices described herein, such as delivery device 100, are configured to receive and dispense a fluid. In some embodiments, this fluid may be a biocompatible polymer composition for facilitating and maintaining hemostasis. In some embodiments, such biocompatible polymer composition may, when delivered to a wound site, adhere thereto and effectively create a physical barrier and wound closure component. In some embodiments, the fluid may be in the form of a gel.

In some embodiments, the fluid may be VETIGEL™.

### 3. Kits

Provided herein, in some embodiments, are kits or articles of manufacture, comprising any of the components described herein. For instance, provided herein is a kit of parts comprising the delivery devices described herein (e.g., delivery device 100 of FIG. 1), and a fluid to be dispensed from the delivery device. In some embodiments, the fluid may be separately packaged from the delivery device. In some embodiments, the delivery device may be pre-filled with the fluid. In some embodiments, the kit may include a sealed package in which the delivery device and the fluid are enclosed.

In some embodiments, the fluid in the kit may be a hemostatic composition. In some embodiments, the fluid may be VETIGEL™.

In some embodiments, the kit may comprise a delivery device as described herein pre-filled with about 5 mL of a fluid. In some embodiments, the kit may comprise a delivery device as described herein pre-filled with about 5 mL of a hemostatic composition. In some embodiments, the kit may comprise a delivery device as described herein pre-filled with about 5 mL of VETIGEL™.

### 4. Methods of Use

In some embodiments, the delivery devices, as described herein, may be used to deliver the fluid present therein to a wound, for example, an external laceration, an abrasion, a burn, an ocular laceration, damage to a parenchymal organ, an internal laceration, a laceration in the gastrointestinal tract, superficial cuts and scrapes, internal bleeding, an arterial bleed, a venous bleed, dental or oral bleeds and incisions, of a subject (e.g., a human or animal subjects including, but not limited to, mammals such as horses, sheep, cattle, hogs, dogs, cats, etc. and marine animals such as whales, dolphins, seals, otters, fish, etc., and reptiles such as turtles). In delivery devices as described herein may also be useful for delivery the fluid to wounds caused unintentionally (such as accidents or unforeseen injuries) as well as those caused intentionally (such as in surgery).

In some embodiments, the delivery devices as described herein may be used to deliver the fluid present therein directly onto a bleeding wound surface. In embodiments in which the fluid comprises a hemostatic composition, said composition may aid in the clotting of the blood at the composition-blood interface. The particular advantages of the delivery devices described herein, such as the large bore size of the applicator region thereof and/or the absence of connector (e.g., a threaded fitting) within the interior of the applicator region, enables the fluid to be rapidly, and uniformly delivered to the wound surface.

Unless otherwise defined, all technical and scientific terms used herein have the same meaning as commonly understood by one of ordinary skill in the art to which this invention belongs.

Throughout the present specification and claims, unless the context requires otherwise, the word “comprise” and variations thereof (e.g., “comprises” and “comprising”) are to be construed in an open, inclusive sense, that is as “including, but not limited to.” Additionally, the singular forms “a,” “an” and “the” include plural referents unless the context clearly dictates otherwise.

Recitation of numeric ranges of values throughout the specification is intended to serve as a shorthand notation of referring individually to each separate value falling within the range inclusive of the values defining the range, and each separate value is incorporated in the specification as it were individually recited herein.

Reference to “about” a value or parameter herein includes (and describes) embodiments that are directed to that value or parameter per se. In some embodiments, the term “about” includes the indicated amount ±10%.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment, but may be in some instances. Furthermore, the particular features, structures, or characteristics may be combined in any suitable manner in one or more embodiments.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

The invention described and claimed herein is not to be limited in scope by the specific embodiments disclosed herein, as these embodiments are intended as illustrations of several aspects of the invention. Indeed, various modifications of the invention in addition to those shown and described herein will become apparent to those skilled in the art from the foregoing description. Such modifications include the substitution of known equivalents for any aspect of the invention in order to achieve the same result in substantially the same way. Such modifications are also intended to fall within the scope of the appended claims.

1. A delivery device, comprising:
a barrel comprising opposing first and second ends, and a first interior chamber positioned therebetween, wherein a diameter of the first interior chamber is from about 8 mm to about 25 mm and the first interior chamber is configured to receive a fluid;
a plunger comprising a stopper, an end portion, and a shaft extending therebetween, wherein the stopper and one or more portions of the shaft are configured to be disposed within the first interior chamber of the barrel, wherein the plunger is configured to move along a longitudinal axis of the barrel to displace fluid disposed therein;
an applicator region proximal to the first end of the barrel, the applicator region comprising a second interior chamber in fluid communication with the first internal chamber of the barrel, and a connector on an exterior surface of the applicator region, wherein a diameter of the second interior chamber of the applicator region is at least 50% of the diameter of the first interior chamber of the barrel, a length of the second interior chamber of the applicator region ranges from about 5 mm to about 20 mm, and the applicator region is configured for needleless application of the fluid; and
a grip region proximal to the second end thereof, and which extends substantially perpendicular to the barrel, wherein a longitudinal length of the grip region is at least approximately two times as long as an outer diameter of the barrel.

2. (canceled)

3. The delivery device of claim 1, wherein the diameter of the second interior chamber of the applicator region is at least about 5 mm.

4. (canceled)

5. The delivery device of claim 1, wherein the diameter of the second interior chamber of the applicator region is from about 6 mm to about 10 mm.

6. (canceled)

7. The delivery device of claim 1, further comprising a cap configured to surround one or more portions of the applicator region, the cap comprising a second connector complementary to the connector of the applicator region.

8. The delivery device of claim 7, wherein the connector of the applicator region comprises a plurality of male threads, and the second connector of the cap comprises a plurality of female threads.

9. The delivery device of claim 1, wherein a cross-sectional area of the end portion of the plunger is larger than a cross-sectional area of the first interior chamber of the barrel.

10. (canceled)

11. The delivery device of claim 1, wherein the stopper of the plunger is deformable such that the stopper of the plunger is moveable within the first interior chamber.

12. The delivery device of claim 1, wherein the stopper further comprises an engaging member resistively contacting or engaging a retaining member at or near the second end of the first interior chamber, the engaging member comprising a protruding formation preventing separation of the plunger from the first interior chamber.

13. A kit comprising the delivery device of claim 1, and a fluid to be dispensed from the delivery device, wherein the fluid is a hemostatic composition.

14-17. (canceled)

18. A delivery device, comprising:
a barrel comprising opposing first and second ends, and a first interior chamber positioned therebetween, wherein a diameter of the first interior chamber is from about 8 mm to about 25 mm;
a fluid disposed within the first interior chamber;
a plunger comprising a stopper, an end portion, and a shaft extending therebetween, wherein the stopper and one or more portions of the shaft are configured to be disposed within the first interior chamber of the barrel, wherein the plunger is configured to move along a longitudinal axis of the barrel to displace the fluid disposed therein; and
an applicator region configured for needleless application of the fluid proximal to the first end of the barrel, the applicator region comprising a second interior chamber in fluid communication with the first internal channel of the barrel, wherein the second interior chamber ranges in length from about 5 mm to about 20 mm and has a diameter at least 50% of the diameter of the first interior chamber, and a connector implemented on an exterior surface of the applicator region, an outer surface of the applicator region solely comprising a threaded surface area accepting a cap configured to surround the applicator region.

19. The delivery device of claim 18, wherein the diameter of the second interior chamber of the applicator region is from 50% to about 90% of the diameter of the first interior chamber of the barrel.

20. (canceled)

21. The delivery device of claim 18, wherein the connector comprises a plurality of threads.

22. The delivery device of claim 18, the cap comprising a second connector complementary to the connector of the applicator region.

23. The delivery device of claim 18, wherein the fluid is a hemostatic composition.

24. (canceled)

25. The delivery device of claim 22, wherein the plunger further comprises one or more engaging members positioned at or near the stopper and contacting one or more retaining members at or near the second end of the first interior chamber, the engaging member comprising a protruding formation preventing separation of the plunger from the first interior chamber.

26. (canceled)

27. The delivery device of claim 1, wherein an entirety of the connector of the applicator region comprises the plurality of male threads, minimizing a length of the applicator region such that the length of the applicator region provides only enough surface area on which the plurality of female threads are disposed.

28-29. (canceled)

30. The delivery device of claim 1, wherein the diameter of the first interior chamber is about 12 mm.

31-32. (canceled)

33. The delivery device of claim 1, wherein the fluid is in the form of a gel.

34-35. (canceled)

36. The delivery device of claim 18, wherein the fluid is a biocompatible polymeric composition in the form of a gel.

37. A delivery device configured for delivery of a gel, the delivery device comprising
a barrel comprising opposing first and second ends, and a first interior chamber positioned therebetween, wherein a diameter of the first interior chamber is from about 8 mm to about 25 mm and the first interior chamber is configured to receive a fluid in the form of a gel;
a plunger comprising a stopper, an end portion, and a shaft extending therebetween, wherein the stopper and one or more portions of the shaft are configured to be disposed within the first interior chamber of the barrel, wherein the plunger is configured to move along a longitudinal axis of the barrel to displace fluid disposed therein;
an applicator region proximal to the first end of the barrel, the applicator region comprising a second interior chamber in fluid communication with the first internal chamber of the barrel, and a connector on an exterior surface of the applicator region, wherein a diameter of the second interior chamber of the applicator region is at least 50% of the diameter of the first interior chamber of the barrel, a length of the second interior chamber of the applicator region ranges from about 5 mm to about 20 mm, and the applicator region is configured for needleless application of the gel; and
a grip region proximal to the second end of the barrel, and which extends substantially perpendicular to the barrel, wherein a longitudinal length of the grip region is at least approximately two times as long as an outer diameter of the barrel.
